(19) United States
(12) Patent Application Publication
(10) Pub. No.: US 2021/0220536 A1
(43) Pub. Date: Jul. 22, 2021

(54) MEDICAL DEVICE AND METHOD FOR OPERATING A MEDICAL DEVICE

(71) Applicant: FRESENIUS MEDICAL CARE DEUTSCHLAND GMBH, Bad Homburg (DE)

(72) Inventor: Lars OFFERMANNS, Leverkusen (DE)

(73) Assignee: FRESENIUS MEDICAL CARE DEUTSCHLAND GMBH, Bad Homburg (DE)

(21) Appl. No.: 15/998,960

(22) PCT Filed: Feb. 16, 2017
(86) PCT No.: PCT/EP2017/000228

§ 371 Date: Jul. 22, 2019

(30) Foreign Application Priority Data
Feb. 17, 2016 (DE) 10 2016 001 868.2

(51) Int. Cl. A61M 1/16

(52) U.S. Cl. CPC A61M 1/1601; A61M 1/1643; A61M 2205/587; A61M 2205/6081; A61M 2205/6054

(57) ABSTRACT

The present invention relates to a medical device having one or more functional units which are configured to interact with one or more disposables in the operation of the device, wherein the medical device furthermore has one or more illuminants, and wherein the device can be operated in different treatment modes, wherein at least one control unit or regulation unit is provided which are configured such that they control the illuminants in dependence on the selected treatment mode of the medical device in which the medical device is or should be operated, with the illuminants being arranged such that the light emanating from them at least partly illuminates the one or more functional units. The invention furthermore relates to a method of operating a medical device.

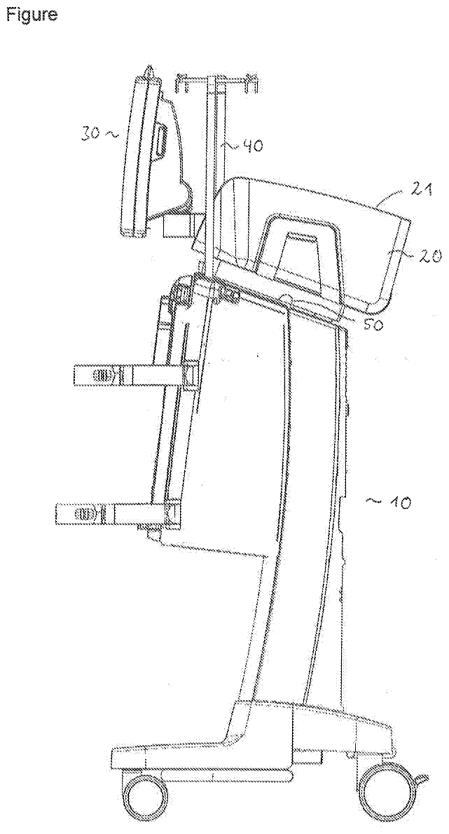

The present invention relates to a medical device, in particular to a blood treatment device, having one or more functional units which interact with one or more disposables in the operation of the device, wherein the medical device furthermore has one or more illuminants, and wherein the device can be operated in different treatment modes.

It is known from the prior art to design dialysis devices with scale pans into which solution pouches are laid within the framework of preparing the device for carrying out a dialysis treatment. The solution pouches remain in the scale pans during the dialysis treatment. The solution pouches are, for example, pouches containing dialyzate and/or substituate. In this respect, it is important for the success of the treatment and for the safety of the patient that the correct solution pouch is placed into the correct scale pan, i.e. the scale pan intended for it.

A medical device is known from DE 10 2013 008 213 A1 in which a part of a device such as the device housing is illuminated, whereby specific information such as filling levels or treatment times, etc. can be displayed.

It is the underlying object of the present invention to further develop a medical device of the initially named kind such that the probability of an incorrect association of a disposable with a functional unit is reduced with respect to arrangements known from the prior art.

This object is achieved by a medical device having the features of claim 1. Provision is accordingly made that the device has at least one control unit or regulation unit which are configured such that they control the illuminants in dependence on the selected treatment mode in which the medical unit is operated, with the illuminants being arranged such that the light emanating from them at least partly illuminates the at least one functional unit.

The illuminants are thus controlled such that at least one property of the light emanating therefrom depends on the selected treatment mode.

It is thus possible, for example, to illuminate one functional unit with a specific illumination color and to illumination another functional unit with a specific illumination color differing therefrom.

The scale pan for the solution pouch containing the dialyzate can thus, for example, be illuminated in a yellow color and the scale pan for the solution pouch containing the substituate solution can be illuminated in a blue color.

The colors in which the one or more functional units are illuminated preferably correspond to the colors or color codes of the disposable which are to interact with the functional units, i.e., for example, to the color codes affixed to the solution pouches.

Any desired property of the light dependent on the treatment mode with which the at least one functional unit is illuminated is covered by the invention.

It is preferably the color of the light, but the brightness, the color depth, the contrast, the kind of illumination, e.g. continuous, intermittent, etc., is also covered by the invention.

The functional unit can be any desired unit which interacts in any desired manner with one or more disposables in the operation of the device and/or in the treatment preparation.

A scale pan or a receiver for a holder for a disposable can, for example, be considered as a functional unit.

The medical device is preferably a dialysis device. However, any other desired medical devices in which disposables, i.e. single-use articles, are used to carry out a treatment are also covered by the invention.

As stated above, the functional unit or units in a preferred embodiment of the invention is/are one or more scale pans for receiving solution pouches whose weight is determined in the course of the treatment by a load cell connected to the scale pan.

The functional unit or units can, for example, be one or more holders or receivers for one or more hose kits, filters or dialyzers.

A particularly good recognizability results when the at least one functional unit is formed as partly completely transparent or translucent. In the case of scale pans, for example, this allows them to be illuminated from below.

An illumination of the functional unit from any desired side is generally conceivable and covered by the invention.

The term “illumination” does not only cover the case that the light is incident onto the functional unit, but also the case that the light emanates from the functional unit in that the illuminants are, for example, arranged in the interior of the functional unit or in that a planar LED or OLED is, for example, affixed to the functional unit.

A further illumination option, for example, comprises coupling the light emanating from a side edge of the functional unit into said functional unit, whereby a good recognizability and an appealing overall visual impression results.

The kind of the illumination can be as desired as long as it is easily recognizable for a user. The illumination can relate to the total functional unit or only to a part thereof. It can, for example, be configured as a uniform illumination or also have a specific form, for example in the form of lettering or of a logo.

As stated above, the illuminants can be configured such that they emit light, for example, in different colors, brightnesses or in different time routines.

The illuminants can, for example, be LEDs, in particular RGB LEDs so that different illumination colors can be produced. Any other desired illuminants such as incandescent lamps, etc, are, however, also covered by the invention.

In a preferred embodiment of the invention, the medical device has detection means by means of which at least one property of the disposable or disposables can be detected.

The property of the disposable can e.g. be color markings, graphical codes such as barcodes or also information which is transmitted to the detection means from passive or active transponders. They can, for example, be affixed to the wrapping or to the housing of the disposable.

It is furthermore conceivable that the device has comparison means which are configured to compare the information detected by the detection means with a desired piece of information. It can be determined at the device side in this manner whether, for example, the correct solution pouch is disposed in the correct scale pan, whether the correct filter is attached in the correct holder and whether the correct hose kit has been inserted, etc.

To provide the user with a monitoring option, the device can have output means by means of which the information detected by the detection means, the result of the comparison carried out in the comparison means or a piece of information based thereon can be output in a manner recognizable for a user of the device. It is thus possible that the information is stored in the device that a specific filter or a specific solution is to be used. If it is found that this is not the case, a corresponding message can be output to the user and, optionally, a further measure can be taken such as that of the putting into operation of the medical device being inhibited for so long until the fault has been remedied.

It is thus possible that the device checks the actual equipping with the disposable or disposables and, optionally, outputs feedback on one or more recognized disposables and/or on incorrect configurations.

The detections means can, for example, be a camera, a means for recognizing RFID tags or other near field detection means which can detect markings of the disposable or disposables at distances of, preferably, <1 m.

The present invention furthermore relates to a system comprising at least one medical device in accordance with one of the claims 1 to 11, wherein the device is equipped with one or more disposables. As stated above, the disposable or disposables can, for example, be solution pouches, hose kits, filters, dialyzers, etc.

The disposables are preferably designed in color in accordance with the treatment mode. It is thus conceivable, for example, that one of the scale pans is illuminated in yellow and the other scale pan of the device is illuminated in blue as part of a hemodiafiltration treatment. The solution pouches to be placed onto the scale pans, which contain dialyzate, on the one hand, and substituate, on the other hand, have a corresponding color design, that is likewise yellow and blue in accordance with the example.

If, for example, a hemodialysis treatment is selected as the treatment mode, if no substituate is thus required, but only dialyzate, the illumination of both scale pans can take place in yellow so that it is immediately visible to the user that solution pouches filled with dialyzate (having a yellow color code) are to be placed onto both scale pans.

The present invention furthermore relates to a method having the features of claim 14.

Provision is accordingly made that the illuminants are controlled in dependence on the selected treatment mode of the medical device in which the medical device is or should be operated, with the illuminants being arranged such that the light emanating from them at least partly illuminates the one or more functional units.

The light is preferably selectively output in different colors, brightnesses or in different time routines.

It is conceivable for one property of the disposable or disposables to be detected, with provision preferably being made that the information detected by the detection means is compared with a piece of desired information.

In a further embodiment of the invention, the information detected by the detection means, the result of the comparison made or a piece of information based thereon is output in a manner recognizable for a user of the device such as on a monitor of the device.

Further details and advantages of the invention will be explained in more detail with reference to an embodiment shown in the drawing.

The only FIGURE shows a dialysis device in accordance with the invention in a schematic view.

The embodiment relates to a dialysis device 10 which can be used or operated in different treatment modes.

The dialysis device can be used in a first treatment mode for an HDF, i.e. for a hemodiafiltration, and in a second mode for an HD, i.e. for a hemodialysis.

A further distinction of the treatment modes can be carried out with respect to the anticoagulant used (calcium or heparin as the coagulant).

The device has two scale pans 20, 21 which are formed from a transparent material such as plastic and which are connected to a scale pan which determines the weight of the solution pouches located in the scale pans permanently or at specific time intervals.

As can be seen from the FIGURE, the scale pans 20, 21 are located above the device body and behind the screen 30 which forms the interface to the user and is, for example, formed as a touch screen so that information can be output and input. In the embodiment, two scale pans 20, 21 are provided which are arranged next to one another and of which only one (scale pan 20) is visible in the side view in accordance with the FIGURE. The second scale pan is indicated by the arrow and by the reference numeral 21.

The reference numeral 40 marks a stand for suspending solution pouches.

RGB LEDs, i.e. LEDs or LED arrays which can selectively output light in red, green, or blue or also in mixed colors, are located beneath or next to the scale pans 20, 21. They are likewise only shown schematically and are marked by the reference numeral 50.

The device has selection means by means of which the user can set the kind of treatment (HDF or HD) and the anticoagulant (citrate or heparin). The selection means can, for example, be push buttons, switches or also fields on the screen 30 configured e.g. as a touch screen.

If, for example, a CVVHDF treatment (CVVHDF=continuous venous-venous hemodiafiltration) is carried out using a citrate-based anticoagulation (Ci-Ca), the illuminants 50 are controlled by means of a control unit such that the first scale pan is illuminated in yellow and the second scale pan is illuminated in blue.

In the case of a CVVHD treatment (CVVHD=continuous venous-venous hemodialysis) using a citrate-based anticoagulation, the illuminants 50 are controlled by means of a control unit such that the first scale pan 20 is illuminated in yellow and the second scale pan 21 is also illuminated in yellow.

If a CVVHD therapy is selected in which heparin anticoagulation is carried out, a switch can be made to an illumination of the first scale pan 20 and of the second scale pan 21 in blue.

The illumination of the pan scales 20, 21 can be selected accordingly before the start of the treatment or also on a change of the treatment. In both cases, it is immediately visible to the user which color-coded solution pouch he has to place into which scale pan 20, 21.

If, in the above-named example, a switch is made from a citrate-based anticoagulation to a heparin-based anticoagulation (or vice versa) so that a change of the solution pouches is necessary, a corresponding change of the illumination color from yellow to blue (or vice versa) takes place.

The illumination color preferably corresponds to the color coding on the solution pouches so that is immediately recognizable for a user which pouch has to be placed onto which scale pan (20, 21).

To largely preclude incorrect treatments, the device can be configured with detection means which are formed to recognize which color coding the solution pouches have and to transmit this information to comparison means. They check whether the detected color coding corresponds to a desired color coding. If this is not the case, it is conceivable that the control unit or regulation unit prevents the carrying out of the treatment or at least outputs a text message to the user so that he recognizes that an incorrect association of the solution pouches with the scale pans 20, 21 has taken place and a change of the solution pouches has to be carried out.

If further solution pouches are to be introduced, the color coding of the RGB LEDS or of other illuminants can be expanded in a simple manner.

1. A medical device having one or more functional units which are configured to interact with one or more disposables in the operation of the device, wherein the medical device furthermore has one or more illuminants, and wherein the device can be operated in different treatment modes, characterized in that at least one control unit or regulation unit is provided which are configured such that they control the illuminants in dependence on the selected treatment mode of the medical device in which the medical device is or should be operated, with the illuminants being arranged such that the light emanating from them at least partly illuminates the one or more functional units.

2. A medical device in accordance with claim 1, characterized in that the medical device is a dialysis device.

3. A medical device in accordance with claim 1, characterized in that the functional unit or units is/are one or more scale pans for receiving solution pouches to be weighed.

4. A medical device in accordance with claim 1, characterized in that the functional unit or units is/are one or more holders or receivers for one or more hose kits, filters or dialyzers.

5. A medical device in accordance with claim 1, characterized in that the at least one functional unit is formed as partly or completely transparent or translucent.

6. A medical device in accordance with claim 1, characterized in that the illuminants are configured such that they selectively emit light in different colors, brightnesses or in different time routines.

7. A medical device in accordance with claim 1, characterized in that the illuminants are configured as LEDs, in particular as RGB LEDs or comprise LEDs and in particular RGB LEDs.

8. A medical device in accordance with claim 1, characterized in that the medical device has detection means by means of which at least one property of the disposable or disposables can be detected.

9. A medical device in accordance with claim 8, characterized in that the medical device has comparison means which are configured to compare information detected by the detection means with a piece of desired information.

10. A medical device in accordance with claim 8, characterized in that the medical device has output means by means of which the information detected by the detection means, the result of the comparison made in the comparison means or a piece of information based thereon can be output in a manner recognizable for a user.

11. A medical device in accordance with claim 8, characterized in that the detection means are one or more cameras, means for recognizing RFID tags or other near field detection means.

12. A system comprising at least one medical device in accordance with claim 1, wherein a medical device is equipped with one or more disposables.

13. A system in accordance with claim 12, characterized in that the disposable or disposables is/are color designed in accordance with the treatment mode.

14. A method of operating a medical device, preferably in accordance with claim 1, or of operating a system which comprises a medical device having one or more disposables, wherein the medical device has one or more functional units which are configured to interact with said one or more disposables in the operation of the device, wherein the medical device furthermore has one or more illuminants, and wherein the device can be operated in different treatment modes, characterized in that the illuminants are controlled in dependence on a selected treatment mode of the medical device in which the medical device is or should be operated, with the illuminants being arranged such that the light emanating from them at least partly illuminates the one or more functional units.

15. A method in accordance with claim 14, characterized in that the light is selectively output in different colors, brightnesses or in different time routines.

16. A method in accordance with claim 14, characterized in that at least one property of the disposable or disposables is detected, with provision preferably being made that the information detected by the detection means is compared with a piece of desired information.

17. A method in accordance with claim 16, characterized in that the information detected by the detection means, the result of the comparison made or a piece of information based thereon is output in a manner recognizable for a user.
